(19) United States
(12) Patent Application Publication
(10) Pub. No.: US 2003/0044442 A1
(43) Pub. Date: Mar. 6, 2003

(54) Controlled breakdown granules

(76) Inventors: Peter W Stanier, Sandbach (GB); Pamela E Baines, Warrington (GB)

Correspondence Address: PILLSBURY WINTHROP, LLP, P.O. BOX 10500, MCLEAN, VA, 22102, US

(21) Appl. No.: 10/220,312

(22) PCT Filed: Feb. 8, 2001
(86) PCT No.: PCT/GB01/00488

(30) Foreign Application Priority Data
Feb. 29, 2000 (GB) 0004723.3

(51) Int. Cl. A61K 9/14; A61K 7/00

(52) U.S. Cl. 424/401; 424/489

(57) ABSTRACT

A granular composition for use in a personal care product comprises at least one water-insoluble inorganic material having a particle size of no more than 50 μm and up to 10 percent by weight based on weight of water-insoluble inorganic material of a non-binding swelling agent, the granules having a particle size greater than about 50 μm and up to about 1000 μm, as measured by sieve analysis, a dry strength such that from about 20 to about 70 percent by weight pass through a 212 μm sieve when subjected to the attrition test defined herein and a wet strength such that less than 12 percent by weight residue remains on a 45 μm sieve after ultrasonification in water for 1 minute with 48 μm amplitude of vibration, and wherein said granules possess sufficient strength so as not to substantially break down when present in said personal care product.

The present invention relates to an inorganic material in granular form useful in personal care products and particularly to an inorganic material possessing controlled breakdown properties. The present invention also relates to personal care products containing said inorganic material.

Exfoliation and cleansing of the skin is an essential element of body care in which particles are used to remove residual make-up and dead cells from the surface of the skin in order to prevent pores clogging. Conventional exfoliants, such as calcium carbonate and the endocarp of apricot seeds have an inherent grittiness and there is a consumer desire to have an exfoliant material which has an initial skin feel which disappears while using the cosmetic product.

Thus, EP-A-670 712 discloses an exfoliating composition including a particulate exfoliating material with a particle size in the range of 0.03 to 3 mm, wherein the particulate material comprises an agglomerated silica having a primary particle size in the range of 0.01-0.2 μm, which is friable and under conditions of use of the composition breaks up into particles having an average size of less than 40 μm.

As disclosed in this document particles with an average size of less than 40 μm do not feel gritty and the average particle size, after break up of the exfoliating particles, is less than 40 μm. Nevertheless, it has been found that, whilst the grittiness is reduced, the particles are still felt by the user as a residue on the skin.

An improved exfoliating material is disclosed in PCT application WO-A-97/30126, this material being capable of breaking down upon ultrasonification. Nevertheless, materials need to possess sufficient strength to withstand the forces experienced during manufacture, transport and formulating and it has been found that break-down granules which meet this criterion do not break down quickly enough for some applications.

There is still, therefore, a need for exfoliating particles which provide the required exfoliating performance, and break down quickly to a point at which they are no longer detected, but also have sufficient dry strength to be handled satisfactorily.

Particles possessing controlled breakdown properties are also useful in other personal care products. For example, there is an increasing desire to produce dental cleaning products, such as toothpastes and gels in which the user perceives a mouth feel related to the abrasives present in the products. However, it is also desirable that this mouth feel is reduced during brushing, providing an indication to the user that sufficient cleansing has been achieved.

According to the present invention there is provided a granular composition for use in a personal care product said granular composition comprising at least one water-insoluble inorganic material having a particle size of no more than 50 μm (preferably no more than 40 μm and more preferably no more than 30 μm) and up to 10 percent by weight based on weight of water-insoluble inorganic material of a non-binding swelling agent, the granules having a particle size greater than about 50 μm and up to about 1000 μm, as measured by sieve analysis, a dry strength such that from about 20 to about 70 percent by weight pass through a 212 μm sieve when subjected to the attrition test defined herein and a wet strength such that less than 12 percent, preferably less than 10 percent, most preferably less than 8 percent, by weight residue remains on a 45 μm sieve after ultrasonification in water for 1 minute with 48 μm amplitude of vibration, and wherein said granules possess sufficient strength so as not to substantially break down when present in said personal care product.

The water-insoluble inorganic material can be chosen from a wide range of materials, including amorphous silicas, aluminas, calcium carbonates, dicalcium phosphate, tribasic calcium phosphates, insoluble sodium metaphosphate, calcium pyrophosphates, hydroxyapatites, perlites, zeolites, magnesium carbonate and pumice.

By “water-insoluble”, we mean a compound with a water solubility of less than 5 g, preferably less than 1 g, per 100 g water at a temperature of 25° C.

A particularly useful form of the inorganic material comprises amorphous silica or a mixture of amorphous silica with another inorganic material. Preferred mixed inorganic material in granular form comprises at least 95 percent by weight of a water-insoluble inorganic material, whereby 5 to 90 percent by weight of the water-insoluble inorganic material is made from a first component having a weight mean particle size of less than 20 μm and an oil absorption capacity of 90 to 145 cm³/100 g, and selected from the group consisting of amorphous silicas, aluminas, calcium carbonates, dicalcium phosphate, tribasic calcium phosphates, insoluble sodium metaphosphate, calcium pyrophosphates, hydroxyapatites, perlites, zeolites, magnesium carbonate and pumice, and 5 to 90 percent by weight of the water-insoluble inorganic material is made from an amorphous silica having a weight mean particle size of below 20 μm and an oil absorption capacity of 150 to 190 cm³/100 g. This form of inorganic material has been found to be particularly suitable for use in skin care products.

A preferred form of this mixture comprises a mixture of two types of silica fulfilling the above criteria, that is, inorganic material in granular form comprising at least 95 percent by weight of particulate silica, whereby 5 to 90 percent by weight of the particulate silica is made from a silica having a weight mean particle size of less than 20 μm and an oil absorption capacity of 90 to 145 cm³/100 g and 5 to 90 percent by weight of the particulate silica is made from an amorphous silica having a weight mean particle size of below 20 μm and an oil absorption capacity of 150 to 190 cm³/100 g. The amorphous silica may be silica gel or precipitated silica.

A further preferred form of the inorganic material in granular form comprises at least 95 percent by weight of a water-insoluble inorganic material, whereby 5 to 90 percent by weight of the water-insoluble inorganic material is made from a first component having a weight mean particle size of less than 20 μm and an oil absorption capacity of 90 to 145 cm³/100 g, and selected from the group consisting of aluminas, calcium carbonates, dicalcium phosphate, tribasic calcium phosphates, insoluble sodium metaphosphate, calcium pyrophosphates, hydroxyapatites, perlites, zeolites, magnesium carbonate and pumice, and 5 to 90 percent by weight of the water-insoluble inorganic material is made from an amorphous silica, having a weight mean particle size of below 20 μm and an oil absorption capacity of 130 to 190 cm³/100 g. This form of inorganic material has also been found to be suitable for use in skin care products.

A preferred mixed inorganic material in granular form which has been found to be suitable for use in dental compositions comprises from 45 to 98 percent by weight of a water-insoluble inorganic material, whereby 10 to 75 percent by weight of the water-insoluble inorganic material is made from a first component having a weight mean particle size of less than 20 μm and an oil absorption capacity of 60 to 180 cm³/100 g, and selected from the group consisting of amorphous silicas, aluminas, calcium carbonates, dicalcium phosphate, tribasic calcium phosphates, insoluble sodium metaphosphate, calcium pyrophosphates, hydroxyapatites, perlites, zeolites, magnesium carbonate and pumice, and 10 to 75 percent by weight of the water-insoluble inorganic material is made from an amorphous silica having a weight mean particle size of below 20 μm and an oil absorption capacity of 200 to 350 cm³/100 g.

The particle size of 50 μm given above for the inorganic material is a maximum particle size. A convenient method of measuring particle size is by using a Malvern Mastersizer, the technique being more fully described hereinafter. This technique produces a measure of particle size distribution and, for the purposes of this invention, the “maximum particle size” of a sample, when measured by this technique, is taken to be the 99 percentile (d₉₉) of this distribution, i.e. 99 percent by weight has a particle size, as determined by Malvern Mastersizer, below the value taken to be the maximum particle size. Preferably, the inorganic material has an average particle size of less than 20 μm, more preferably less than 15 μm. Usually, the inorganic particles will have an average size greater than 1 μm, and, preferably, greater than 5 μm.

The oil absorption is an important parameter for silica which is of use in forming the granular compositions of the invention but the appropriate value of this parameter depends, to some extent, on the personal care product for which the granular composition is designed. Preferred forms of silica for use in granular compositions for skin care products have an oil absorption in the range 90 to 190 cm³/100 g, and preferred forms of silica for use in granular compositions for dental products have an oil absorption in the range 130 to 350 cm³/100 g. When two forms of silica are used to produce the granular composition, as described hereinbefore, the oil absorption of the mixed material will normally be arranged to be within the appropriate range as described for a single form of silica.

The swelling agent is frequently a compound which swells in the presence of water. Consequently, preferred water-insoluble inorganic materials used to prepare the granular compositions of the invention contain a relatively low water content. Preferably, water content of the inorganic materials is less than 20 percent by weight, more preferably less than 15 percent by weight and, most preferably, less than 10 percent by weight. The method used to determine water content of the inorganic material should be the most appropriate method for the material in question. For silica, water content is determined by measuring the total volatile content at 1000° C., as described in more detail hereinafter.

The swelling agent which is present in the granules according to the invention is “non-binding”. By this is meant that the granules do not rely upon the presence of the swelling agent for their dry strength (i.e. the swelling agent is not used for binding the individual water-insoluble inorganic particles to one another). Some of the swelling agents are materials which can be used as binders, but do not do so in the granular compositions of this invention. Although the invention is not limited by the following theory, it is believed that the dry strength of the granular composition is derived from the interparticulate bonding of the inorganic material and the swelling agent exists as dry particles in the dry granular compositions. When the granular compositions are incorporated into a personal care product, the swelling agent interacts with a component of the product, such as water, and the particles of the swelling agent are caused to swell within the granular compositions, leading to a weakening of the structure of these compositions. Generally, granular compositions according to this invention have a dry strength which is similar to granular compositions prepared from the same inorganic material but in the absence of the swelling agent.

Consequently, suitable swelling agents comprise materials capable of swelling on contact with a component of a personal care product. Particularly useful swelling agents are organic polymers that swell in the presence of water and are known as “super-absorbents”. Typical such organic polymers may, for example, be selected from the group consisting of sodium starch glycolates, sodium polyacrylates, cross-linked sodium carboxymethyl celluloses and mixtures thereof. Alternatively, inorganic materials such as swelling clays (for example Laponite™) can be used. The swelling agent preferably has a water swelling capacity of at least 10 ml/g, more preferably 15 ml/g and most preferably at least 20 ml/g, typically at least 30 ml/g (e.g. 50 ml/g or greater). Desirably, the average particle size of the swelling agent is less than 100 μm, more preferably less than 50 μm, prior to swelling.

The amount of swelling agent present in the granular compositions is up to 10 percent by weight based on weight of water-insoluble inorganic material. The amount used can be influenced by the intended use for the personal care product. Where the personal care product is a skin care product, this product will generally contain a relatively high proportion of water and the initial feel of the particles on the skin should be relatively soft. Consequently, the amount of swelling agent present is preferably from 0.1 up to 5.0 percent by weight and more preferably up to 2.0 percent by weight. Typically, the amount used is in the range 0.5 to 1.8 percent by weight based on weight of inorganic material. However, a dental cleansing product may require a harder granular composition, to provide an initial “bite”, and, generally, the principal contact with water is during use. Consequently, it is generally necessary to use more swelling agent to achieve the desired objectives. For dental applications, the amount of swelling agent present is preferably in the range 0.5 to 10 percent by weight based on weight of inorganic material and more preferably in the range 1 to 5 percent by weight.

The particle size of the granules, as measured by sieve analysis, is in the range 50 to 1000 μm. The preferred size depends, to some extent, on the nature of the personal care product. For skin care products, the preferred particle size is in the range 70 to 1000 μm and a useful product has a particle size in the range 70 to 500 μm, with a weight average particle size in the range 250 to 500 μm. When the granules are designed to be visible in the skin care product, the preferred particle size is in the range 200 to 1000 μm with an average particle size in the range 400 to 700 μm. In dental products, the particle size is preferably in the range 50 to 600 μm and more preferably in the range 100 to 450 μm, with a weight average particle size in the range 150 to 350 μm.

The dry strength of the granules is determined by an attrition test in which the granules are subjected to high shear and which is described more fully hereinafter. The granules need to possess an adequate strength to facilitate processing and transport without degradation and to remain substantially intact when incorporated in the personal care product but should be sufficiently weak to break down in use. This controlled break-down produces a desired tactile feel, for example a massaging feeling without a sensation of abrasiveness. The dry strength is related to the degree of attrition in the test, as measured by the proportion passing through a 212 μm sieve after attrition. The intended use for the personal care product will influence the preferred dry strength as measured by the amount passing through the sieve. For skin care products, the preferred range is 40 to 70 percent by weight passing through a 212 μm sieve after attrition and, more preferably, 40 to 60 percent by weight passes through a 212 μm after attrition. For dental products, preferred granules have a dry strength such that from 20 to 40 percent, more preferably from 30 to 40 percent by weight, passes through a 212 μm sieve.

The wet strength of the granules is a measure of the ease with which they break down in use. This is determined by measuring particle size after ultrasonification and is described in more detail hereinafter.

The granules of the invention are also required to remain substantially intact when incorporated into the personal care product, in order to achieve the desired tactile properties. This is achieved by a suitable adjustment of the amount of swelling agent and the dry strength, taking into account the amount of water or other swelling solvent present in the personal care product. The particles can often be visibly discernible in the product (especially in transparent products when the particles are also coloured), which provides a visible demonstration that they have remained intact. Alternatively, the integrity of the particles can be determined using the Mechanical Strength test described in detail below. In this test the personal care product is mixed with propylene glycol and the resulting mixture subjected to ultrasonification, after which the granules are separated and dried and the residue on a 45 μm sieve is determined. A substantially intact granular composition has a residue on a 45 μm sieve greater than 50 percent by weight on this test. Preferably the residue on the 45 μm sieve is at least 55 percent by weight and, more preferably, the residue is at least 60 percent by weight on a 45 μm sieve.

According to a second aspect of the invention, a method of preparing a granular composition for use in a personal care product comprises agglomerating a mixture of at least one water-insoluble inorganic material having a particle size of no more than 50 μm (preferably no more than 40 μm and more preferably no more than 30 μm) and up to 10 percent by weight based on weight of water-insoluble inorganic material of a swelling agent so as to produce a granular composition, the granules of which have a particle size greater than about 50 μm and up to about 1000 μm, as measured by sieve analysis, a dry strength such that from about 20 to about 70 percent by weight pass through a 212 μm sieve when subjected to the attrition test defined herein and a wet strength such that less than 12 percent, preferably less than 10 percent, most preferably less than 8 percent, by weight residue remains on a 45 μm sieve after ultrasonification in water for 1 minute with 48 μm amplitude of vibration, said granules possessing sufficient strength so as not to substantially break down when present in said personal care product and wherein said swelling agent does not act as a binder.

Agglomeration of the inorganic material and swelling agent may be achieved by any suitable agglomeration method. It is important to avoid agglomeration conditions which lead to any significant swelling of the swelling agent, since such conditions would lead to the production of a weaker granule in the dry state.

Preferably, the agglomeration is achieved using a roller compactor and suitable compactors include the Fitzpatrick Chilsonater commercially available from the Fitzpatrick Company, and the Alexanderwerk roller compactor, commercially available from Alexanderwerk GmbH. Operating conditions are selected on the compactor so that the resultant granule containing inorganic material and the swelling agent has an attrition value (measurement of dry strength) which is within the specified range. The selection of appropriate conditions to achieve the desired dry strength is well within the abilities of a person skilled in the art of granulation of inorganic materials. One important parameter is the pressure applied to the inorganic material by the rollers. Usually, this is less than 10 MPa and is preferably less than 5 MPa. Generally, a pressure of at least 1 MPa is necessary to produce a dry strength within the range specified for the granular compositions of the invention.

The material to be tested for attrition value needs to be within the specified size range. This may be achieved by subjecting the agglomerates emerging from the compactor to a grinding/comminution device, such as a hammer mill. The resulting particles are screened to provide particles in the size range from about 50 to about 1000 μm.

Surprisingly, utilising effective compacting conditions, agglomerates can be prepared containing the swelling agent that are strong enough to withstand normal factory handling encountered in the production of personal care products and which break down, on use, particularly in contact with water (where the swelling agent is swelled by water), into particles that are small enough to be tactilely undetectable but, nevertheless, produce a desirable tactile sensation initially.

If coloured granules are required, then suitable colouring material can be added to the composition of the granule, without affecting the strength of the granule. The principal requirement is that the colouring material is suitable for use in personal care products, but, where the swelling agent is swelled by water, colouring materials containing water should be avoided. Suitable materials include solid pigments and laked dyes. Typical materials include pigment powders sold under the Hostaperm trade name, Cosmetic Pink RC 01 (D & C Red No 30) supplied by Clariant, Ultramarine Grade 54 or Ariabel Green supplied by Warner Jenkinson and Sicomet P74160 or Sicomet P74260 supplied by BASF.

According to a further aspect of the invention, a personal care product comprises a granular composition comprising at least one water-insoluble inorganic material having a particle size of no more than 50 μm (preferably no more than 40 μm and more preferably no more than 30 μm) and up to 10 percent by weight based on weight of water-insoluble inorganic material of a non-binding swelling agent, the granules having a particle size greater than about 50 μm and up to about 1000 μm, as measured by sieve analysis, a dry strength such that from about 20 to about 70 percent by weight pass through a 212 μm sieve when subjected to the attrition test defined herein and a wet strength such that less than 12 percent, preferably less than 10 percent, most preferably less than 8 percent, by weight residue remains on a 45 μm sieve after ultrasonification in water for 1 minute with 48 μm amplitude of vibration, and wherein said granules possess sufficient strength so as not to substantially break down in said personal care product.

Preferably, the personal care product is in the form of a liquid, a structured liquid, a paste, an emulsion or a multiple emulsion. By suitable adjustment of the solid to liquid ratio, and the viscosity of the liquid phase, the product may take any physical form from a thick paste or gel to a low viscosity liquid. The invention also includes personal care products wherein the product is essentially dry, particularly where such products are used in an aqueous environment where the swelling agent can swell rapidly and assist in the controlled breakdown of the granules (e.g. dental powders).

In the personal care products of the present invention, the level of inorganic material in granular form may be from 0.5 to 20 percent by weight, preferably 1 to 10 percent, more preferably 1 to 5 percent and even more preferably 3 to 5 percent by weight. In certain applications, the granular material is used to create a visual effect, but any exfoliation should be minimal. In such circumstances, the preferred amount of granular composition in the personal care product is in the range 0.5 to 1.0 percent by weight.

The personal care product of the invention may contain one or more additional components depending on the end use of the product, typical end uses being personal wash off products, for example shower and bath washing products, facial cleansers and shampoos, and dental products in the form of a toothpaste, gel, cream or liquid, of the opaque or transparent variety.

Cleaning compositions, including dental cleaning compositions, also comprise one or more surfactants, preferably selected from anionic, cationic, nonionic, amphoteric and zwitterionic surfactants and mixtures thereof. The surfactants may be present in a total amount of from 1 to 50 percent by weight, preferably from about 2 to 30 percent by weight.

Commonly, water is another component of the personal care products of the present invention and may be present in an amount from 5 to 90 percent by weight, preferably from 10 to 80 percent by weight, more preferably from 20 to 80 percent by weight.

It is preferred that one or more thickening or suspending agents are included in the personal care products of this invention in order that the inorganic material in granular form remains stably dispersed throughout the composition. These agents may be present in the compositions in a total amount of from 0.1 to about 60 percent by weight, preferably about 0.1 to 10 percent by weight, depending on the nature of the agents.

The personal care products of the invention may also contain other components conventionally found in personal care products for skin, hair or teeth, etc. Examples include perfumes, flavouring agents, artificial sweeteners, pearlescing agents, opacifiers, pigments and colourings, preservatives, moisturising agents, antibacterial agents, anticaries agents, anti-hypersensitivity agents and pH adjusting agents.

Personal care products in accordance with the present invention may be made by conventional methods of preparing such products. If suspension is through surfactant lamellar phase formation, however, it is preferable that the granular composition is incorporated in the formulation of the product prior to the formation of the lamellar phase which stabilises the dispersed particles, in order that the granular composition be successfully and stably incorporated therein. Alternatively, for creams lotions, gels and pastes, the base composition may be prepared by mixing the base ingredients, with addition of thickener or suspending agent if used, followed by low shear mixing of the pre-prepared granular composition.

It is important that in the preparation of personal care products in accordance with the present invention that any mixing be done at sufficiently low shear that the inorganic material in granular form does not experience forces sufficiently great to cause substantial amounts of the granules to fracture.

The granular composition of the current invention is characterised by employing a number of tests. The methods for carrying out these tests are described in detail below.

Oil Absorption

The oil absorption is determined by the ASTM spatula rub-out method (American Society Of Test Material Standards D, 281). The test is based on the principle of mixing linseed oil with the inorganic material by rubbing with a spatula on a smooth surface until a stiff putty-like paste is formed which will not break or separate when it is cut with a spatula. The oil absorption is then calculated from the amount of oil (O grams) used by applying the following equation:

Oil absorption=O×100/Wt. of inorganic material sample in grams

i.e. Oil absorption=grams of oil used/100 grams of inorganic material

Weight Mean Particle Size

The weight mean particle size of the water-insoluble inorganic material is determined using a Malvern Mastersizer model X, with a 45 mm lens and MS15 sample presentation unit. This instrument, made by Malvern Instruments, Malvern, Worcestershire, uses the principle of Mie scattering, utilising a low power He/Ne laser. Before measurement the sample is dispersed ultrasonically in water for 7 minutes to form an aqueous suspension. This suspension is stirred before it is subjected to the measurement procedure outlined in the instruction manual for the instrument, utilising a 45 mm lens in the detector system. The Malvern Mastersizer measures the weight particle size distribution of the inorganic material or reference material. The weight mean particle size (d₅₀) or 50 percentile, the 10 percentile (d₁₀), the 90 percentile (d₉₀) and the 99 percentile (d₉₉) are readily obtained from the data generated by the instrument.

Particle Size Distribution by Sieve Analysis

Particle size distribution of the granular composition is carried out using sieve analysis. 100 g of the sample is placed on the top sieve of a series of BS sieves, at approximately 100 μm intervals between 100 and 1500 μm. The sieves are arranged in order with the finest at the bottom and the coarsest at the top of the stack. The sieves are placed in a mechanical vibrator, for example, Inclyno Mechanical Sieve Shaker by Pascall Engineering Co Ltd., covered with a lid and shaken for 10 minutes. Each sieve fraction is accurately weighed and the results calculated:

% residue=(weight of residue×100)/weight of sample.

Total Volatile Matter

This test is applicable to silica and is determined by heating the silica to constant weight in a furnace at 1000° C. The Total Volatile Matter is given by the loss in weight expressed as a percentage of the weight of silica after heating.

Dry strength (Attrition Test)

The dry strength is determined by a method based on attrition of the granules in a high-shear mixer. A control is first carried out to determine the percentage by weight of fines (<212 μm) already present or generated by the sieving process. For the control approx. 20 grams of accurately weighed granules is sieved for 10 minutes over a 212 μm sieve on a laboratory sieve shaker. The percentage by weight of material passing the 212 μm sieve is recorded. For the test, a Sirman C6vv food processor, manufactured by Sirman s.n.c, Marsango, Italy and available from Metcalfe Catering Equipment, Blaenau Ffestiniog, Gwynedd, Wales was used. This processor is powered by a 350 watt (0.5 HP) motor and has a 5.5 l capacity bowl. The bowl diameter is 20.3 cm and the rotor is equipped with two curved, chamfered blades 18 cm in diameter and manufactured from AISI 420 stainless steel. 20.0 grams of granules are placed in the food processor and the processor turned on at maximum speed (2100 revs./min.) for one minute. The sample after attrition is sieved for 10 minutes, as before, and the percentage by weight passing the 212 μm sieve measured. The percentage by weight already less than 212 μm (the control) is subtracted to obtain the attrition value.

Wet Strength

Granule breakdown characterisation was carried out using a Microson XL2020 Sonicator programmable ultrasonic liquid processor, manufactured by Misonix Inc. Farmingdale, N.Y. and supplied in the UK by Labcaire Systems Ltd, Avon.

The Microson XL2020 Sonicator ultrasonic processor has a maximum of 550 watts output with a 20 kHz converter and is fitted with a 19 mm (¾ inch) tapped horn. The processor has variable amplitude control and a microprocessor controlled digital timer integrated with a Pulsar cycle timer with power output and elapsed time displays. The piezoelectric converter transforms electrical energy to mechanical energy at a frequency of 20 kHz. Oscillation of piezoelectric crystals is transmitted and focused by a titanium disrupter horn that radiates energy into the liquid being treated. A phenomenon known as cavitation, the formation and collapse of microscopic vapour bubbles generated by the strong sound waves produces a shearing and tearing action. Almost all of the activity takes place just in front of the probe tip.

The generator provides high voltage pulses of energy at 20 kHz and adjusts for varying load conditions, such as viscosity and temperature. It senses impedance change and increases or decreases power to the probe tip automatically. The probe is a medium intensity horn for processing volumes between 25 and 500 ml. The maximum amplitude at the tip of the probe is 60 μm (setting 10). Hence, sonicator processors operating at output control setting 8 have 48 μm of amplitude (peak to peak amplitude of the radiating face of the tip) at the tip of the probe.

Therefore, there is a linear relationship between the output control knob (or amplitude adjustment knob) and the amplitude at the tip of the probe, i.e. 6 μm of amplitude per control knob setting. The generator draws energy accordingly to maintain a constant amplitude at the tip for a given output control setting. This is displayed on the percentage output power meter and is energy in watts (i.e. output=%/100×550 watts available=y watts delivered).

A paper given by Mr. S. Berliner, (Director, Technical Services, Heat Systems-Ultrasonics Inc.) at the 9th Annual Technical Symposium of the Ultrasonic Industry Association, entitled “Application of Ultrasonic Processors (Power vs Intensity in Sonification)” provides further detailed information of the principles involved in this experimental technique.

Procedure:

A 250 ml Pyrex beaker is insulated and fitted with a lid with a 19 mm hole in the centre to accommodate the ultrasonic probe and a 3 mm hole to the side to accommodate a temperature probe. Into the insulated beaker, 199 g of water is weighed, and maintained at a constant temperature of 50° C. using a magnetic stirrer hotplate equipped with a temperature sensor (Heidolph MR3003 magnetic stirrer hotplate with a stainless steel PT-100 temperature sensor and stirrer speed indicator (in rpm), obtainable from Orme Scientific, Manchester). 1 g of the granule to be tested is added. The ultrasonic probe is immersed to a depth of 16 mm into the liquid and the temperature sensor is inserted into the liquid to continuously monitor temperature. The beaker contents are stirred on setting 3 (approximately 300 rpm), for 2 minutes prior to introducing ultrasonification.

The Sonicator ultrasonic processor is switched on and information on processing time and pulsed mode programmed, as required.

Cavitation is introduced to the system by turning the output control knob to the desired amplitude setting, whilst the temperature profile is closely monitored. The percentage power output required to maintain the amplitude at the tip is also recorded, according to the setting. Stirring is continued through the cavitation process. When the cavitation process is complete, the stirrer is switched off and the magnetic stirrer bar is removed. Manual stirring is continued with a spatula to maintain dispersion and the inorganic particle dispersion is poured through a 45 μm sieve. Any residue in the beaker is washed through the sieve, using half the amount of initial water. The sieve is then dried to constant weight in an oven at 105° C. The residue which remains on top of the 45 μm sieve is then weighed and expressed as a percentage of the initial weight of inorganic granule. The greater the amount retained on the sieve, the stronger the agglomerate strength of the granule and the more difficult it is to break down. It has been found that, for a granule to satisfactorily break down in personal care products, it will have less than 12%, preferably less than 10%, most preferably less than 8% by weight, residue on a 45 μm sieve after ultrasonification on setting 8 (48 μm amplitude) for a period of 1 minute.

Mechanical Strength

This test is used to demonstrate that the granular compositions according to the invention do not break down substantially in the personal care products in which they are useful. The test uses ultrasonification employing the same principles as are applied in the Wet Strength test described above.

A known weight of a personal care product (preferably about 24.50 g) containing a known weight of inorganic granular composition (preferably about 0.50 g) is hand-stirred in a Sterilin container with a nominal volume of 55 ml to ensure that the granules are well-dispersed and allowed to equilibrate for approximately 12 hours. The equilibrated formulation is poured into a 250 ml beaker equipped as described in the Wet Strength test above with continuous hand stirring with a spatula. The Sterilin container is rinsed into the beaker with propylene glycol and the final weight of the beaker contents is made up to 175 g with propylene glycol. A magnetic stirrer bar is introduced into the beaker and the beaker is placed on a magnetic stirrer hotplate equipped with a temperature sensor (Heidolph MR3003 magnetic stirrer hotplate with a stainless steel PT-100 temperature sensor and stirrer speed indicator (in rpm), obtainable from Orme Scientific, Manchester). The beaker contents are stirred on setting 3 (approximately 300 rpm), the ultrasonic probe is immersed to a depth of 16 mm into the liquid and the temperature sensor is immersed into the liquid to continuously monitor temperature. The beaker contents are stirred for 30 seconds and the Sonicator ultrasonic processor is switched on and information on processing time and pulsed mode is programmed, as required. Cavitation is introduced to the system by turning the output control knob to the desired amplitude setting, whilst the temperature profile is closely monitored. The percentage power output required to maintain the amplitude at the tip is also recorded, according to the setting.

When the cavitation process is complete, the stirrer is switched off and the magnetic stirrer bar is removed. The beaker contents are left to stand overnight in order that the particles settle prior to quantification of the residue remaining above 45 μm. The beaker is transferred to a fume cupboard and the propylene glycol is decanted from the particulate sediment at the bottom of the beaker. Isopropyl alcohol (approximately 25 cm³) is added as a wash and the resultant mixture stirred with a spatula. The particles are allowed to settle and the isopropyl alcohol is decanted and the wash repeated twice to ensure that the propylene glycol is removed. Manual stirring is continued with a spatula to maintain dispersion and the inorganic particle dispersion is poured through a 45 μm sieve. Any residue in the beaker is washed through the sieve, using half the amount of initial isopropyl alcohol. The sieve is then dried to constant weight in an oven at 105° C. The residue which remains on top of the 45 μm sieve is then weighed and expressed as a percentage of the initial weight of inorganic granule. The greater the amount retained on the sieve, the less the granule has broken down on addition to the personal care product. Ideally, 100 percent of the initial granular material should be retained on the 45 μm sieve.

The invention is illustrated by the following, non-limiting examples.

### EXAMPLES

### General Procedure for Preparing Samples

Silica granular compositions were prepared by blending two silicas and, except for the comparative example, a swelling agent, as dry ingredients in a high shear mixer known as a Pek mixer (George Tweedy & Co of Preston—28 lb S.A. Machine) and compacting on a roller compactor (Alexanderwerk WP50—manufactured by Alexanderwerk AG, D 5630 Remscheid 1, Germany). The two silicas were Sorbosil AC39 (low to medium structure) and Neosyl AC (medium structure), both available from Crosfield Limited, which were blended in a 9:1 Sorbosil: Neosyl ratio by weight.

The silicas had the following properties.

| Property | | | Sorbosil AC39 | Neosyl AC |
|---|---|---|---|---|
| Oil Absorption (cm³/100 g) | | | 125 | 155 |
| | Particle Size | d₁₀ | 3.2 | 3.7 |
| | Distribution | d₅₀ | 11.3 | 11.9 |
| | (μm) | d₉₀ | 31.7 | 38.1 |

Silica and swelling agent (when used) were blended together, in the appropriate proportions, in a Pek mixer for 30 minutes. 2 kg of material, prepared as described above, was compacted by feeding into an Alexanderwerk roller compactor, fitted with a sintered block vacuum deaeration system. The settings used for the preparation of the Examples in this invention were: roller speed 2, screw feeder 4, vacuum 0.8, stirrer speed 2. The roller pressure used was 2.5 MPa.

The compacted material from the compactor was fed into a granulator, which forms part of the machine, and forced through a 1.2 mm mesh. The resulting granules were then screened by gently forcing them through a 1000 μm screen and then sieving at 500 μm to adjust the particle size distribution.

Examples using the above procedure were prepared as shown in Table 1 below in which the amount of swelling agent is expressed as a percentage by weight of the amount of silica.

TABLE 1

| Example Number | Swelling agent/amount¹ |
|---|---|
| A | None |
| 1 | Nilyn XL-90²/0.75% |
| 2 | Nilyn XL-90²/1.5% |
| 3 | Nilyn XL-90²/5% |
| 4 | Aquasorb A500³/0.75% |
| 5 | Aquasorb A500³/1.5% |
| 6 | Vivastar P5000⁴/1.5% |
| 7 | Laponite⁵/1.5% |

¹Expressed as percentage of weight of silica.
²Nilyn XL-90 is croscarmellose sodium available from FMC Corporation, Philadelphia, USA
³Aquasorb A500 is a sodium carboxymethyl cellulose available form Hercules Limited, Salford, UK.
⁴Vivastar P5000 is a sodium starch glycolate obtainable from J. Rettenmaier & Sohne, Germany.
⁵Laponite is a swelling clay available from Laporte, Widnes, UK.

The examples were subjected to a Dry Strength Test as described hereinbefore and the results are shown in Table 2 below.

TABLE 2

| | % by weight passing a 212 μm |
|---|---|
| Example | sieve |
| A | 51.3 |
| 1 | 65.6 |
| 2 | 56.6 |
| 3 | 60.3 |
| 4 | 59.6 |
| 5 | 59.5 |
| 6 | 56.9 |
| 7 | 51.6 |

The granular compositions were incorporated into a personal care base formulation containing 80% by weight water. It is believed that the effect of this formulation on the granules will be similar to the effect of a fully formulated personal wash product such as a shower, bath or hair wash product.

| Ingredient | % by weight |
|---|---|
| Sodium Laureth sulphate (70%)* | 27.00 |
| Carbomer** | 1.00 |
| Water | 72.00 |

*Elfan NS 243S (conc.), available from Akzo.
**Carbomer Ultrez 10, available from B.F. Goodrich.

The formulation was prepared by slowly sifting the Carbomer with continuous stirring into the water, continuing the stirring for a further 30 minutes to ensure complete hydration, adding the sodium laureth sulphate and mixing until dispersed.

24.5 g of the base was weighed into a Sterilin pot with a nominal volume of 55 ml to which 0.50 g of the granule to be tested was added to give a final granule loading of 2.0% by weight in the base formulation. The formulation was left overnight to allow the granules to equilibrate before the Mechanical Strength was assessed using the test described hereinbefore. The results are given in Table 3 below where the residue on a 45 μm sieve, as a percentage of the original weight of granular composition, is reported.

TABLE 3

| Example | Residue on 45 μm sieve |
|---|---|
| A | 70 |
| 1 | 72 |
| 2 | 64 |
| 3 | 60 |
| 4 | 66 |
| 6 | 62 |
| 7 | 70 |

It is believed that the integrity of compositions described in Table 3 above will be more readily retained in personal care base formulations containing less water than that used above.

The Wet strength of the examples was measured using the test described hereinbefore. The results (residue on a 45 μm sieve as a percentage of the original weight of granule) are given in Table 4 below.

TABLE 4

| | Residue on 45 μm sieve |
|---|---|
| Example | (wet test) |
| A | 17.0 |
| 1 | 7.0 |
| 2 | 6.0 |
| 3 | 6.0 |
| 5 | 5.0 |
| 6 | 7.0 |
| 7 | 10.0 |

1. A granular composition for use in a personal care product said granular composition comprising at least one water-insoluble inorganic material having a particle size of no more than 50 μm and up to 10 percent by weight based on weight of water-insoluble inorganic material of a non-binding swelling agent, the granules having a particle size greater than 50 μm and up to 1000 μm, as measured by sieve analysis, a dry strength such that from 20 percent to 70 percent by weight pass through a 212 μm sieve when subjected to the attrition test defined herein and a wet strength such that less than 12 percent by weight residue remains on a 45 μm sieve after ultrasonification in water for 1 minute with 48 μm amplitude of vibration, and wherein said granules possess sufficient strength so as not to substantially break down when present in said personal care product.

2. A granular composition according to claim 1 characterised in that the water-insoluble inorganic material is amorphous silica, alumina, calcium carbonate, dicalcium phosphate, tribasic calcium phosphate, insoluble sodium metaphosphate, calcium pyrophosphate, hydroxyapatite, perlite, a zeolite, magnesium carbonate or pumice.

3. A granular composition according to claim 1 or 2 characterised in that the water-insoluble inorganic material is a mixed inorganic material in granular form comprising at least 95 percent by weight of a water-insoluble inorganic material, whereby 5 percent to 90 percent by weight of the water-insoluble inorganic material is made from a first component having a weight mean particle size of less than 20 μm and an oil absorption capacity of 90 cm³/100 g to 145 cm³/100 g, and selected from the group consisting of amorphous silicas, aluminas, calcium carbonates, dicalcium phosphate, tribasic calcium phosphates, insoluble sodium metaphosphate, calcium pyrophosphates, hydroxyapatites, perlites, zeolites, magnesium carbonate and pumice, and 5 percent to 90 percent by weight of the water-insoluble inorganic material is made from an amorphous silica having a weight mean particle size of below 20 μm and an oil absorption capacity of 150 cm³/100 g to 190 cm³/100 g.

4. A granular composition according to claim 1, 2 or 3 characterised in that the water-insoluble inorganic material comprises at least 95 percent by weight of particulate silica, whereby 5 percent to 90 percent by weight of the particulate silica is made from a silica, having a weight mean particle size of less than 20 μm and an oil absorption capacity of 90 cm³/100 g to 145 cm³/100 g and 5 percent to 90 percent by weight of the particulate silica is made from an amorphous silica, having a weight mean particle size of below 20 μm and an oil absorption capacity of 150 cm³/100 g to 190 cm³/100 g.

5. A granular composition according to claim 1 or 2 characterised in that the water-insoluble inorganic material is a mixed inorganic material in granular form comprising at least 95 percent by weight of a water-insoluble inorganic material, whereby 5 percent to 90 percent by weight of the water-insoluble inorganic material is made from a first component having a weight mean particle size of less than 20 μm and an oil absorption capacity of 90 cm³/100 g to 145 cm³/100 g, and selected from the group consisting of aluminas, calcium carbonates, dicalcium phosphate, tribasic calcium phosphates, insoluble sodium metaphosphate, calcium pyrophosphates, hydroxyapatites, perlites, zeolites, magnesium carbonate and pumice, and 5 percent to 90 percent by weight of the water-insoluble inorganic material is made from an amorphous silica, having a weight mean particle size of below 20 μm and an oil absorption capacity of 130 cm³/100 g to 190 cm³/100 g.

6. A granular composition according to claim 1 or 2 characterised in that the water-insoluble inorganic material is a mixed inorganic material in granular form comprising from 45 to 98 percent by weight of a water-insoluble inorganic material, whereby 10 to 75 percent by weight of the water-insoluble inorganic material is made from a first component having a weight mean particle size of less than 20 μm and an oil absorption capacity of 60 to 180 cm³/100 g, and selected from the group consisting of amorphous silicas, aluminas, calcium carbonates, dicalcium phosphate, tribasic calcium phosphates, insoluble sodium metaphosphate, calcium pyrophosphates, hydroxyapatites, perlites, zeolites, magnesium carbonate and pumice, and 10 to 75 percent by weight of the water-insoluble inorganic material is made from an amorphous silica having a weight mean particle size of below 20 μm and an oil absorption capacity of 200 to 350 cm³/100 g.

7. A granular composition according to any one of the preceding claims characterised in that the water-insoluble inorganic material has an average particle size of less than 20 μm.

8. A granular composition according to any one of claims 1 to 4 or 7 characterised in that the water-insoluble inorganic material is silica having an oil absorption in the range 90 cm³/100 g to 190 cm³/100 g.

9. A granular composition according to any one of claims 1 to 4 or 7 characterised in that the water-insoluble inorganic material is silica having an oil absorption capacity of 130 cm³/100 g to 350 cm³/100 g.

10. A granular composition according to any one of the preceding claims characterised in that the water-insoluble inorganic material used to prepare the granular composition has a water content less than 20 percent by weight.

11. A granular composition according to any one of the preceding claims characterised in that the swelling agent is a swelling clay or an organic polymer selected from the group consisting of sodium starch glycolates, sodium polyacrylates, cross-linked sodium carboxymethyl celluloses, and mixtures thereof.

12. A granular composition according to any one of the preceding claims characterised in that the swelling agent has a water swelling capacity of at least 10 cm³/g.

13. A granular composition according to any one of the preceding claims characterised in that the swelling agent is present in an amount from 0.1 percent up to 2.0 percent by weight based on weight of water-insoluble inorganic material.

14. A granular composition according to any one of the preceding claims characterised in that the granular material has an average particle size in the range 250 μm to 500 μm.

15. A granular composition according to any one of claims 1 to 13 characterised in that the granular material has an average particle size in the range 150 μm to 350 μm.

16. A granular composition according to any one of the preceding claims characterised in that the granular composition has a dry strength such that from 40 percent to 70 percent by weight passes through a 212 μm sieve when subjected to the attrition test defined herein.

17. A granular composition according to any one of claims 1 to 15 characterised in that the granular composition has a dry strength such that from 20 percent to 40 percent by weight passes through a 212 μm sieve when subjected to the attrition test defined herein.

18. A granular composition according to any one of the preceding claims characterised in that the granules have a residue greater than 50 percent by weight on a 45 μm sieve when tested according to the mechanical strength test defined herein.

19. A method of preparing a granular composition for use in a personal care product comprising agglomerating a mixture of at least one water-insoluble inorganic material having a particle size of no more than 50 μm and up to 10 percent by weight based on weight of water-insoluble inorganic material of a swelling agent so as to produce a granular composition, the granules of which have a particle size greater than 50 μm and up to 1000 μm, as measured by sieve analysis, a dry strength such that from 20 percent to 70 percent by weight pass through a 212 μm sieve when subjected to the attrition test defined herein and a wet strength such that less than 12 percent by weight residue remains on a 45 μm sieve after ultrasonification in water for 1 minute with 48 μm amplitude of vibration, said granules possessing sufficient strength so as not to substantially break down when present in said personal care product and wherein said swelling agent does not act as a binder.

20. A method according to claim 19 characterised in that the granules are formed by using a roller compactor.

21. A method according to claim 20 characterised in that the roller compactor is operated at a roller pressure of less than 10 MPa.

22. A personal care product comprising a granular composition comprising at least one water-insoluble inorganic material having a particle size of no more than 50 μm and up to 10 percent by weight based on weight of water-insoluble inorganic material of a non-binding swelling agent, the granules having a particle size greater than 50 μm and up to 1000 μm, as measured by sieve analysis, a dry strength such that from 20 percent to 70 percent by weight pass through a 212 μm sieve when subjected to the attrition test defined herein and a wet strength such that less than 12 percent by weight residue remains on a 45 μm sieve after ultrasonification in water for 1 minute with 48 μm amplitude of vibration, and wherein said granules possess sufficient strength so as not to substantially break down in said personal care product.

23. A personal care product according to claim 22 characterised in that the water-insoluble inorganic material in granular form is present in an amount of from 0.5 percent to 20 percent by weight of the personal care product.

24. A personal care product according to claim 22 or 23 characterised in that from 1 percent to 50 percent by weight of at least one surfactant is present in the personal care product.

25. A personal care product according to any one of claims 22 to 24 characterised in that from 5 percent to 90 percent by weight of water is present in the personal care product.

26. A personal care product according to any one of claims 22 to 25 characterised in that from 0.1 percent to 60 percent by weight of at least one thickening or suspending agent is present in the personal care product.
